(19) United States
(12) Patent Application Publication
(10) Pub. No.: US 2022/0093276 A1
(43) Pub. Date: Mar. 24, 2022

(54) Machine Learning-Based Prediction of Covid-19 Risk Score for Census Tract-Level Communities

(71) Applicant: XY.Health Inc., Cambridge, MA (US)

(72) Inventors: Chirag J. PATEL, Boston, MA (US); Arjun K. MANRAI, North Easton, MA (US); Andrew Shaun DEONARINE, Watertown, MA (US); Genevieve LYONS, Boston, MA (US); Chirag LAKHANI, Cambridge, MA (US); Jerod PARRENT, Cambridge, MA (US)

(73) Assignee: XY.Health Inc., Cambridge, MA (US)

(21) Appl. No.: 17/483,680

(22) Filed: Sep. 23, 2021

Related U.S. Application Data
(60) Provisional application No. 63/083,002, filed on Sep. 24, 2020.
(60) Provisional application No. 63/113,770, filed on Nov. 13, 2020.

(51) Int. Cl. G16H 50/80; G06N 5/00; G06N 20/20; G16H 50/30

(52) U.S. Cl. CPC G16H 50/80; G16H 50/30; G06N 20/20; G06N 5/003

(57) ABSTRACT

The technology disclosed relates to a system and method for predicting chronic disease outcome for census tract-level communities at risk for COVID-19 related complications. The system can access satellite image data of built environment for census tract-level communities and merge the image data with respective chronic disease prevalence data per census tract-level community. This merging of image data with chronic disease prevalence data results in a high-dimensional image space. The system includes logic to identify principal components forming a basis of the high-dimensional image space. A subset of the principal components is selected that cumulatively explain at least fifty percent of the explained variance. The system includes logic to calculate COVID-19 risk score as a weighted combination of the selected principal components. The COVID-19 risk score is provided to public health policy decision makers for use in public health policy decisions.

### PRIORITY APPLICATION

This application claims the benefit of U.S. Patent Application No. 63/083,002, entitled “MACHINE LEARNING-BASED PREDICTION OF COVID-19 RISK SCORE FOR CENSUS TRACT-LEVEL COMMUNITIES,” filed on Sep. 24, 2020 (Attorney Docket No. XYAI 1002-1) and claims the benefit of U.S. Patent Application No. 63/113,770 entitled “MACHINE LEARNING-BASED PREDICTION OF COVID-19 RISK SCORE FOR CENSUS TRACT-LEVEL COMMUNITIES,” filed on Nov. 13, 2020 (Attorney Docket No. XYAI 1002-2). The provisional application is incorporated by reference for all purposes.

### FIELD OF THE TECHNOLOGY DISCLOSED

The technology disclosed relates to use of machine learning techniques to predict risk for chronic disease outcome from COVID-19 related complications.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

COVID-19 has impacted lives of people living in every region of the world. The economic and public health impact of COVID-19 have been unprecedented. Millions of people have been infected and hundreds of thousands have lost their lives. Millions have lost their livelihood. Governments of countries around the world are putting in huge amounts of money to protect their populations from the impact of COVID-19. It is, however, not obvious how public health, social, economic, and other conditions in communities impact the spread of virus. We have observed that certain countries, cities, and communities had a very high infection rates as compared to other countries, cities, and communities. Such information can be important for public health decision makers when deploying resources to protect their communities.

An opportunity arises to develop a system that can analyze public health, economic, and social factors to predict the impact level of COVID-19 on a community.

### BRIEF DESCRIPTION OF THE DRAWINGS

FIG. 1A illustrates an architectural-level schematic of a system to calculate COVID-19 risk score using chronic disease prevalence data and built environment images.

FIG. 1B presents a high-level overview of the technology disclosed illustrating inputs, processing components and outputs.

FIG. 2A presents high-level components of principal component analyzer of FIG. 1A.

FIG. 2B illustrates dimensionality reduction and identification of principal components of high dimensional images by rescaling and flattening.

FIG. 2C illustrates process steps for principal component analysis including using explained variance to select principal components with high cumulative variance.

FIG. 3 is an architectural-level schematic of a system for predicting impact of sociodemographic variables on COVID-19 risk score.

FIG. 4 illustrates training of a random forest predictor and application of the trained random forest to predict impact level of sociodemographic variables.

FIG. 5 is a block diagram of a computer system that can be used to implement the systems of FIGS. 1 and 3.

FIG. 6A presents per-census tract prevalence (along X-axis) of health indicators (along Y-axis).

FIG. 6B presents correlation of health indicators across 27,968 census tracts.

FIG. 7 presents median prevalence within a city versus the interquartile range (IQR) of prevalence of health indicators for top three cities with largest IQR.

FIG. 8 presents variables corresponding to the top two principal components of health indicators in the United States.

FIG. 9 presents incidence rate ratios for a multivariate model to predict COVID-19 deaths.

FIG. 10 presents deep-learning based prediction capability of chronic diseases and the COVID-19 risk score.

FIG. 11 presents satellite views of Zip codes and census tracts with highest and lowest COVID-19 Risk Scores in New York City.

FIG. 12 presents graphical illustration of correlation of death rates and COVID-19 Risk Score.

FIG. 13 presents city-level Median COVID-19 Risk Score versus difference in 75^{th} percentile vs. 25th percentile COVID-19 Risk Score.

FIG. 14 presents an example COVID-19 Community Risk Score web-based dashboard.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

### Introduction

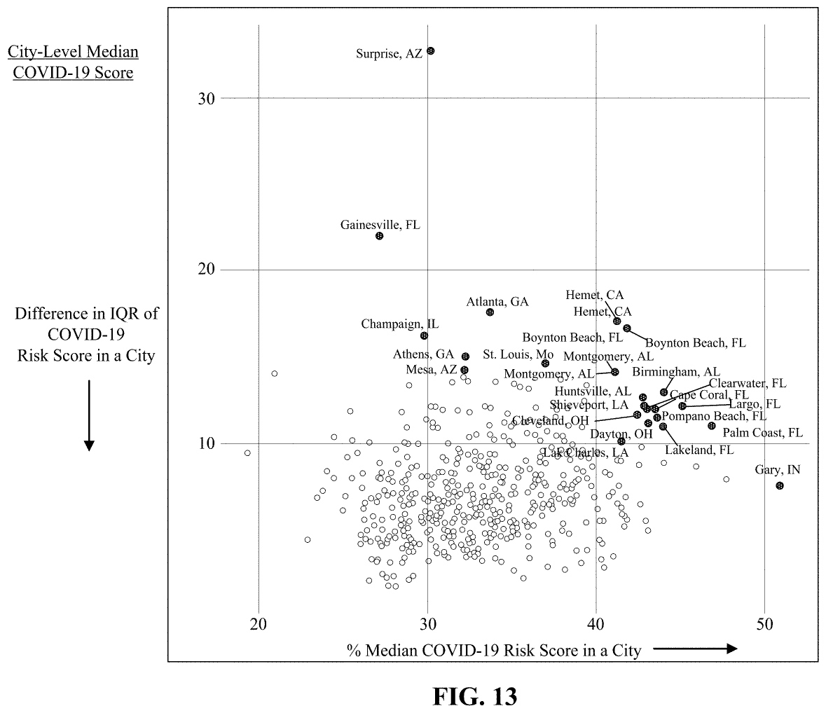

COVID-19 has emerged as a major threat to public health. While it is clear that older individuals with existing comorbidities are at highest risk for complications due to the infection such as hospitalization and death, there is a lack information on what communities are at highest risk for complications. Further, emerging data also suggest that COVID-19 infection and complication is a disease of disparity. Currently, most information collected at a county level obscures local risk complex interactions between social determinants of health, such as clinical comorbidities, the built environment, and demographic factors. The technology disclosed calculates a Census-tract-level COVID-19 Community Risk Score that summarizes the complex co-morbidity and demographic patterns of small communities. The technology disclosed shows how the COVID-19 risk score varies among the many Census tracts per city. A city-level Median COVID-19 score can also be calculated as shown in FIG. 13. This information is important for public health planning and resource allocation. The technology disclosed integrates satellite imagery and census-tract level social determinants of health information and includes a machine learning-based predictor of COVID-19 Community Risk, explaining almost 90% of the Risk Score in the United States in held-out data (R2 of 0.87). We evaluate the technology disclosed using data from-May 2020 and to September 2020 hotspot of COVID-19 epidemic, New York City, and associate the Risk Score with Zip code-level COVID-19 related deaths. We find the COVID-19 Risk Score is associated with a 2-fold greater risk for COVID-19 related death in certain neighborhoods of New York City. We present deployment of the COVID-19 Risk Score with an application programming interface and a browsable dashboard for use by 500 Cities in the United States.

COVID-19 has disrupted major world economies and overwhelmed hospital intensive care units (ICUs) around the world. The virus spread throughout the United States and killed hundreds of thousands of Americans. Even under widespread lockdowns and cessation of normal economic activities, millions of people have been infected in the United States. Many regions have started to reopen local economies or their borders. It remains unclear how weather and climate affect the spread.

Emerging from the case-series and epidemiological surveillance data from the United States (CDC COVID-19 Response Team, “Severe Outcomes Among Patients with Coronavirus Disease) and around the world (Grasselli et al., 2020 “Baseline Characteristics and Outcomes of 1591 Patients Infected with SARS-CoV-2 Admitted to ICUs of the Lombardy Region, Italy”), the following risk factors for COVID-19 related outcomes have been identified: hospitalization, ICU admission, old age, impaired lung function, and cardiometabolic-related diseases (e.g., diabetes, heart disease, stroke) and obesity. Case-series and epidemiological surveillance data from the United States and around the world show that risk factors for COVID-19-related outcomes, such as hospitalization, ICU admission, and death include older age, male sex, impaired lung function, and cardiometabolic-related diseases (e.g., diabetes, heart disease, stroke) and obesity. In the United States, these factors are known to “cluster” in geographies, such as southeast states and counties, and are exacerbated by socio-demographic conditions known as the “social determinants of health” (e.g., in chronic disease and in COVID-19). Other factors of social determinants, such as the built environment and air pollution have tentatively been associated with COVID-19 infection and complications, but it is unclear how to prioritize these associations for complication prevention.

Even within these macro-scale hotspots, prevalent chronic diseases and their risk factors for COVID-19 are geographically heterogenous and vary per unit of geography, including within and across states, counties, and even cities. It is unclear how the heterogeneity of community-based risk—or prevalence of diseases at a census tract-level (median population sizes of 3000 to 5000 individuals) is related to COVID-19 risk. We demonstrate how to calculate a census tract-level COVID-19 Community Risk Score. This score summarizes the complex co-morbidity and demographic patterns of small communities at the census tract, county, and state levels into a single number.

First, we show how the COVID-19 risk score varies per city and illustrate how county-level estimates may obscure identification of specific regions at high risk which is important for resource allocation. Second, we deploy two emerging approaches in machine learning to trace built environment and sociodemographic predictors of COVID-19 community risk. To map the built environment, we use deep learning to query features from satellite images which are common to those used in navigation to build a predictor for COVID-19 community risk score. We also demonstrate how social determinants of health are strongly correlated and predict the COVID-19 Community Risk Score. Finally, we illustrate application of the technology disclosed on one of the hotspots of COVID-19 epidemic, New York City, to show how the COVID-19 community risk score is associated with Zip code-level COVID-19 related deaths independent of social determinants of health. We deploy the COVID-19 risk score with an application programming interface (API) and a browsable dashboard. We present prevalence and heterogeneity of COVID-19 associated co-morbidities and risk factors across 500 cities in the United States.

### Environment

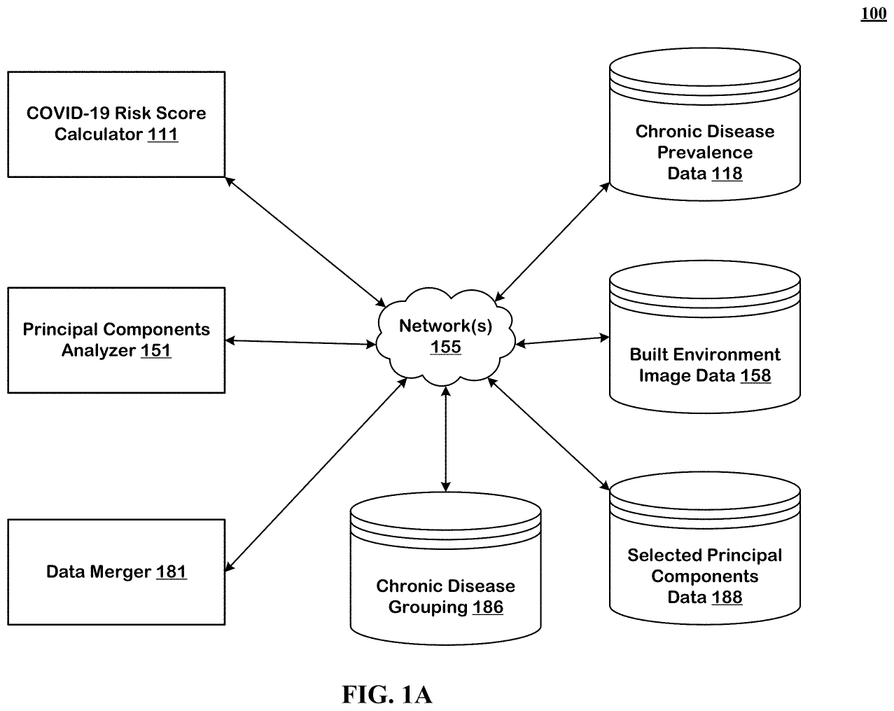

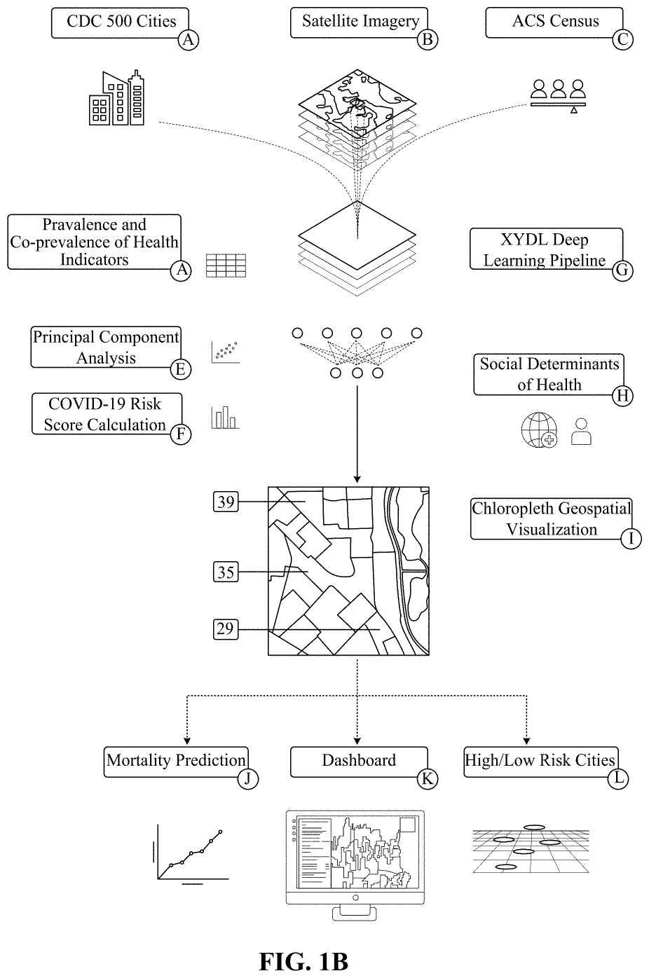

We describe a system for calculating census tract-level COVID-19 community risk score. The system is described with reference to FIG. 1A showing an architectural level schematic of a system in accordance with an implementation. Because FIG. 1A is an architectural diagram, certain details are intentionally omitted to improve the clarity of the description. The discussion of FIG. 1A is organized as follows. First, the elements of the figure are described, followed by their interconnection. Then, the use of the elements in the system is described in greater detail.

FIG. 1A includes the system 100. This paragraph names labeled parts of system 100. The system includes a COVID-19 risk score calculator 111, a principal component analyzer 151, a data merger 181, a chronic disease prevalence database 118, a built environment image database 158, a selected principal components database 188, a chronic disease grouping database 186, and a network(s) 155.

The technology disclosed can use satellite image data of built environment for census tract-level communities for calculating the COVID-19 risk score. The data merger 181 can merge the image data for census tract-level communities with respective chronic disease prevalence data per census tract-level community. The disease prevalence data can include chronic disease prevalence data for a variety of chronic diseases. The system can group chronic disease in categories, e.g., cardiometabolic diseases, cancerous diseases, joint inflammation diseases, etc. Examples of chronic diseases in cardiometabolic diseases category include, diabetes, kidney disease, heart disease, stroke, etc. All types of cancers can be included in the cancerous diseases category. The joint inflammation diseases category can include different types arthritis. The system can include other types of chronic diseases, comorbidities or risk factors. For example, the system can include comorbidities or risk factors for cardiometabolic diseases or impaired lung function such as smoking, obesity, high blood pressure, high cholesterol, kidney disease, asthma, chronic pulmonary obstructive disorder, etc. The system can include comorbidities or risk factors that may be indicative of drug use that might impair immune systems such as, cancer, arthritis, blood pressure drug use, etc. The system can store one or more groupings of chronic diseases in the chronic disease grouping database 186. Chronic disease prevalence data can be stored in chronic disease prevalence database 118.

The system includes a COVID-19 risk score calculator 111 that can calculate a score for census tract-level communities. In other implementations, the system can include a COVID-19 risk score calculator that can calculate COVID-19 risk score for larger communities than census tract-level communities. The system includes a principal component analyzer 151 that can identify principal components of high-dimensional merged data output from the data merger 181. The data merger can merge chronic disease prevalence data with the built environment image data obtained from satellite imagery. The built environment image data is stored in the built environment image database 158. The merged data is in the high-dimensional image space. The principal component analyzer 151 includes the logic to identify the principal components of merged image data in high-dimensional image space and select a subset of principal components. The subset of principal components can be used to project the high-dimensional merged image data to a low-dimensional feature subspace. The selected principal components in the subset have a high cumulative explained variance. The explained variance indicates how much information (or variance) can be attributed to a principal component. The COVID-19 risk score calculator can calculate the COVID-19 risk score for census tract-level communities using a weighted combination of the selected principal components. The system can store selected principal components in the selected principal components database 188.

Completing the description of FIG. 1A, the components of the system 100, described above, are all coupled in communication with the network(s) 155. The actual communication path can be point-to-point over public and/or private networks. The communications can occur over a variety of networks, e.g., private networks, VPN, MPLS circuit, or Internet, and can use appropriate application programming interfaces (APIs) and data interchange formats, e.g., Representational State Transfer (REST), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, point-to-point network, star network, token ring network, hub network, Internet, inclusive of the mobile Internet, via protocols such as EDGE, 3G, 4G LTE, Wi-Fi and WiMAX. The engines or system components of FIG. 1A are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications.

FIG. 1B presents a high-level overview of the technology disclosed. The inputs to the system are labeled as CDC 500 Cities (A), Satellite Imagery (B), and ACS Census data (C). The processing components of the system are labeled as Prevalence and Co-prevalence of health indicators (D), Principal Component Analysis (E), COVID-19 Risk Score Calculation (F), XYDL Deep Learning Pipeline (G), and Social Determinants of Health (H). The system can include a user interface display component labeled as Choropleth Geospatial Visualization (I). Example outputs from the system are labeled as Mortality Prediction (J), Dashboard (K), and High/Low Risk Cities (L). In other implementations, additional inputs, processing components and outputs can be included.

Dimensionality Reduction using Principal Component Analysis

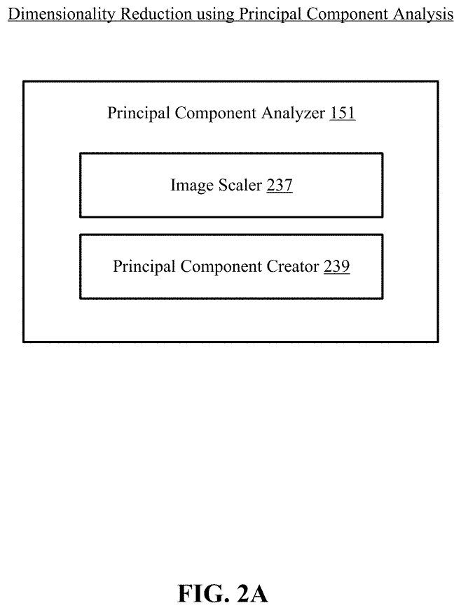

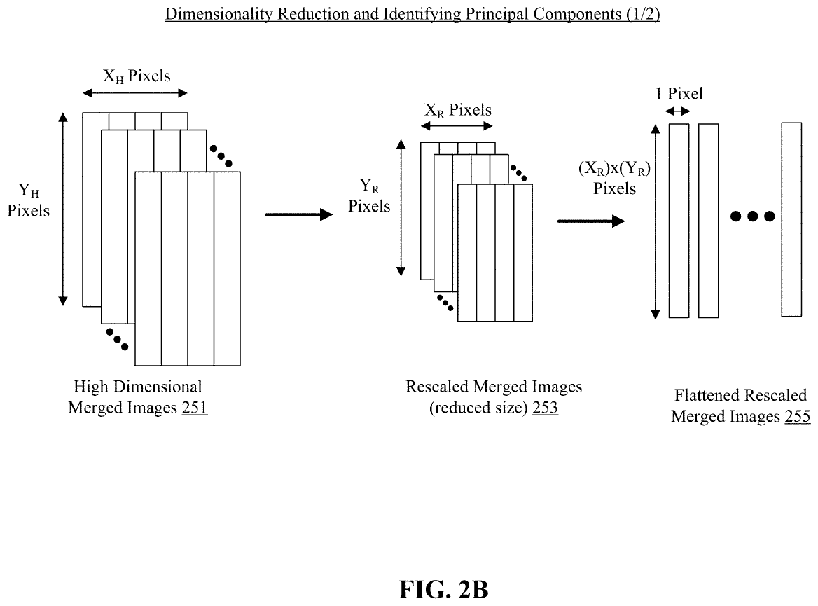

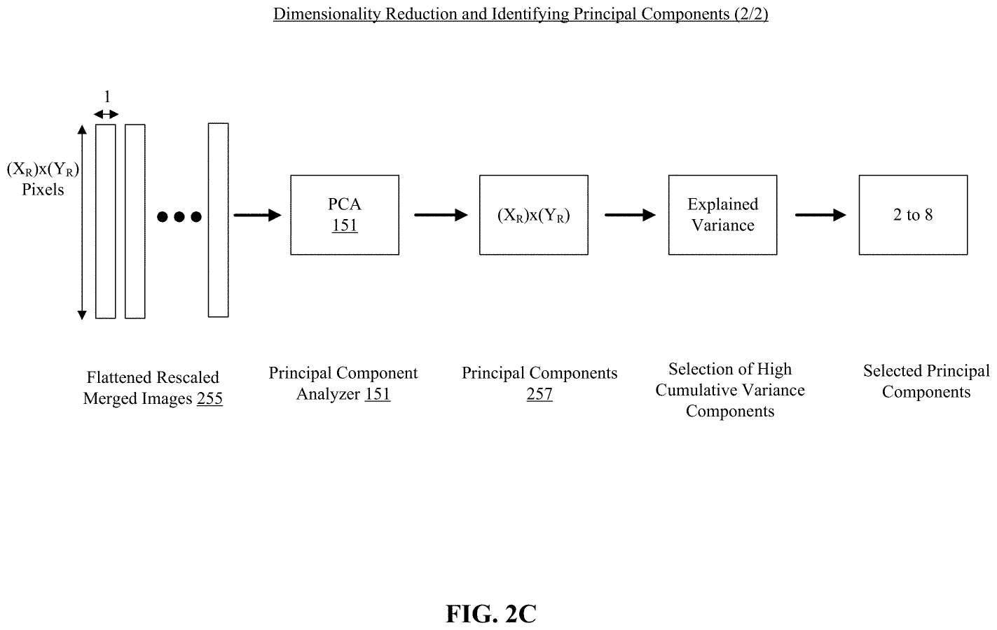

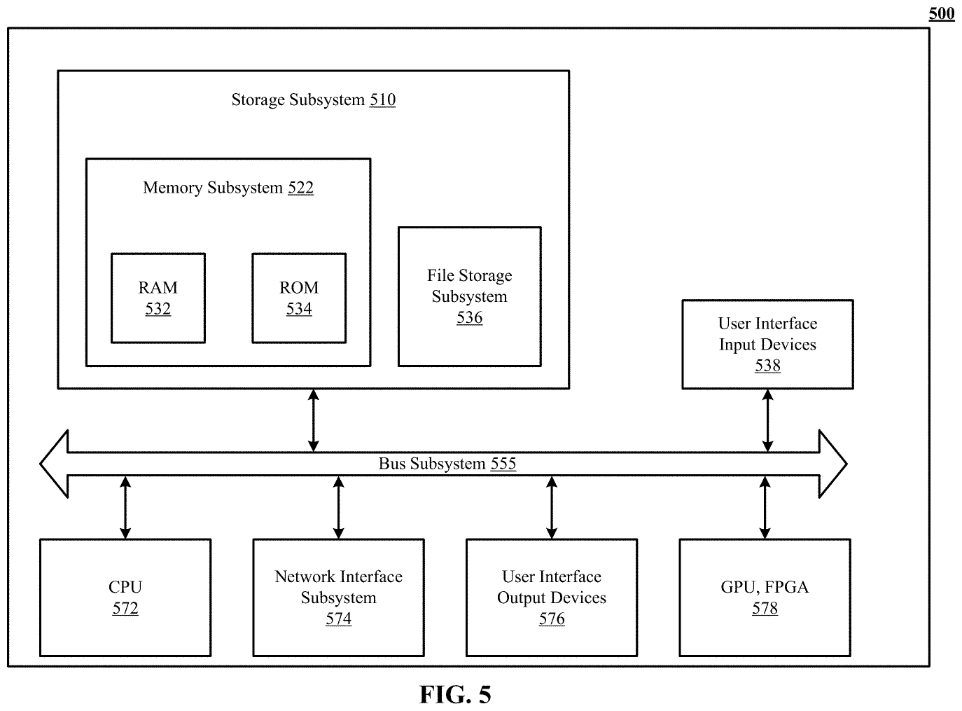

FIG. 2A is a high-level block diagram of components of principal component analyzer 151 which is used for implementing dimensionality reduction. These components are computer implemented using a variety of different computer systems as presented below in description of FIG. 5. The illustrated components can be merged or further separated, when implemented. Principal component analyzer 151 comprises of an image scaler 237 and a principal component creator 239. In the following sections, we present further details of the implementation of these components.

### Principal Component Analyzer

This image processing technique is evolved from facial recognition by Eigen face analysis. One approach to forming an Eigen basis is principal component analysis (PCA). The principal component analyzer 151 applies PCA to merged images. The image scaler component 237 can resize (or rescale) the merged images. Scaling reduces size of merged images so that they can be processed in a computationally efficient manner by the principal component analyzer 151. We present details of these components in the following sections.

Image Scaler

High resolution satellite images can be scaled to reduce the resolution for further analysis. In one instance, images can be reduced in resolution up to 20 times the original resolution of satellite images. In other implementation, images can be reduced in the range of 4 to 25 times the original resolution. The principal component analyzer 151 can process images without rescaling but it can increase the computational cost and time required for processing.

The technology disclosed can apply a variety of interpolation techniques to reduce the size of the production images. In one implementation, bilinear interpolation can be used to reduce size of the merged images. Linear interpolation is a method of curve fitting using linear polynomials to construct new data points with the range of a discrete set of known data points. Bilinear interpolation is an extension of linear interpolation for interpolating functions of two variables (e.g., x and y) on a two-dimensional grid. Bilinear interpolation is performed using linear interpolation first in one direction and then again in a second direction. Although each step is linear in the sampled values and in the position, the interpolation as a whole is not linear but rather quadratic in the sample location. Other interpolation techniques can also be used for reducing the size of the section images (rescaling) such as nearest-neighbor interpolation and resampling using pixel area relation. The technology disclosed can also apply other techniques to reduce the resolution of merged images.

Principal Component Creator

The image processing technique applied to section images to generate input features for classifiers is evolved from facial recognition by Eigen face analysis. From tens of thousands of labeled images, a linear basis of image components is identified. One approach to forming the basis of Eigen images is principal component analysis (PCA). A set B of elements (vectors) in a vector space Vis called a basis, if every element of V may be written in a unique way as a linear combination of elements of B. Equivalently, B is a basis if its elements are linearly independent, and every element of Vis a linear combination of elements of B. A vector space can have several basis. However, all basis have the same number of elements, called the dimension of the vector space.

PCA is often used to reduce the dimensions of a d-dimensional dataset by projecting it onto a k-dimensional subspace where k<d. For example, a resized labeled image in our training database describes a vector of dimension d=X_{H}×Y_{H} pixels which is a high-dimensional space. In other words, the image is a point in X_{H}×Y_{H} high-dimensional space. Eigen space-based approaches approximate the image vectors with lower dimension feature vectors. The main supposition behind this technique is that the image space given by the feature vectors has a lower dimension than the image space given by the number of pixels in the image and that the recognition of images can be performed in this reduced space. Merged satellite images of areas in census tracts, being similar in overall configuration, will not be randomly distributed in this huge space and thus can be described by a relatively low dimensional subspace. The PCA technique finds vectors that best account for the distribution of merged satellite images within the entire image space. These vectors define the subspace of images which is also referred to as “image space”. In our implementation, each vector describes a X_{R}×Y_{R} pixels image (after rescaling to reduce size) and is a linear combination of images in the training data. In the following text, we present details of how principal component analysis (PCA) can be used to create the basis of Eigen images.

The PCA-based analysis of labeled training images can comprise of the following five steps.

### Step 1: Accessing Multi-Dimensional Correlated Data

The first step in application of PCA is to access high-dimensional data. A high dimensional image can have dimension of X_{H} by Y_{H} pixels. High-dimensional merged images are shown by a label 251 in FIG. 2B. High-dimensional merged images are reduced in size resulting in images of size X_{R} by Y_{R} pixels. Each resized image of X_{R}×Y_{R} pixels resolution is represented as a point in a X_{R}×Y_{R} dimensional space, one dimension per pixel. Therefore, the technology disclosed can handle images in a range of high to low resolution. The size of the training data set is expected to increase as we collect more satellite image data. The rescaled merged images 253 are reduced in size as shown in FIG. 2B. Finally, the system can flatten the rescaled merged images 255 as shown in FIG. 2B. The flattened rescaled merged images 255 are given as input to the principal component analyzer 151 as shown in FIG. 2C. We now present the process steps performed by the principal component analyzer in steps 2 to 4 below.

### Step 2: Standardization of the Data

Standardization (or Z-score normalization) is the process of rescaling the features so that they have properties of a Gaussian distribution with mean equal to zero or μ=0 and standard deviation from the mean equal to 1 or σ=1. Standardization is performed to build features that have similar ranges to each other. Standard score of an image can be calculated by subtracting the mean (image) from the image and dividing the result by standard deviation. As PCA yields a feature subspace that maximizes the variance along the axes, it helps to standardize the data so that it is centered across the axes.

### Step 3: Computing Covariance Matrix

The covariance matrix is a d×d matrix of d-dimensional space where each element represents covariance between two features. The covariance of two features measures their tendency to vary together. The variation is the average of the squared deviation of a feature from its mean. Covariance is the average of the products of deviations of feature values from their means. Consider feature k and feature j. Let {x(1, j), x(2, j), x(i, j)} be a set of i examples of feature j, and let {x(1, k), x(2, k), x(i, k)} be a set of i examples of feature k. Similarly, let \tilde{x}ⱼ the mean of feature j and \tilde{x}ₖ be the mean of feature k. The covariance of feature j and feature k is calculated as follows:

$${σ}_{j k} = \frac{1}{n - 1} {\sum }_{i = 1}^{n} ( x ( i , j ) - {\overline{x}}_{j} ) ( x ( i , k ) - {\overline{x}}_{k} ) \qquad ( 1 )$$

We can express the calculation of the covariance matrix via the following matrix equation:

$$\sum = \frac{1}{n - 1} ( {( X - \overline{x} )}^{T} ( X - \overline{x} ) ) \qquad ( 2 )$$

Where the mean vector can be represented as:

$$\overline{x} = \frac{1}{n} {\sum }_{i = 1}^{n} {x}_{i} .$$

The mean vector is a d-dimensional vector where each value in this vector represents the sample mean of a feature column in the training dataset. The covariance value σⱼₖ can vary between the −(σᵢⱼ)(σᵢₖ) i.e., inverse linear correlation to +(σᵢⱼ)(σᵢₖ) linear correlation. When there is no dependency between two features the value of σⱼₖ is zero.

### Step 4: Calculating Eigenvectors and Eigenvalues

The eigenvectors and eigenvalues of a covariance matrix represent the core of PCA. The eigenvectors (or principal components) determine the directions of the new feature space and the eigenvalues determine their magnitudes. In other words, eigenvalues explain the variance of the data along the axes of the new feature space. Eigen decomposition is a method of matrix factorization by representing the matrix using its eigenvectors and eigenvalues. An eigenvector is defined as a vector that only changes by a scalar when linear transformation is applied to it. If A is a matrix that represents the linear transformation, v is the eigenvector and λ is the corresponding eigenvalue, it can be expressed as Av=λv. A square matrix can have as many eigenvectors as it has dimensions. If we represent all eigenvectors as columns of a matrix V and corresponding eigenvalues as entries of a diagonal matrix L, the above equation can be represented as AV=VL. In case of a covariance matrix all eigenvectors are orthogonal to each other and are the principal components of the new feature space.

### Step 5: Using Explained Variance to Select Basis for Eigen Images

The above step can result in (X_{R})×(Y_{R}) principal components 257 (FIG. 2C) for our implementation which is equal to the dimension of the feature space. An eigenpair consists of the eigenvector and the scalar eigenvalue. We can sort the eigen pairs based on eigenvalues and use a metric referred to as “explained variance” to create a basis of eigen images. The explained variance indicates how much information (or variance) can be attributed to each of the principal component. We can plot the results of explained measure values on a two-dimensional graph. The sorted principal components are represented along x-axis. A graph can be plotted indicating cumulative explained variance. The first m components that represent a major portion of the variance can be selected.

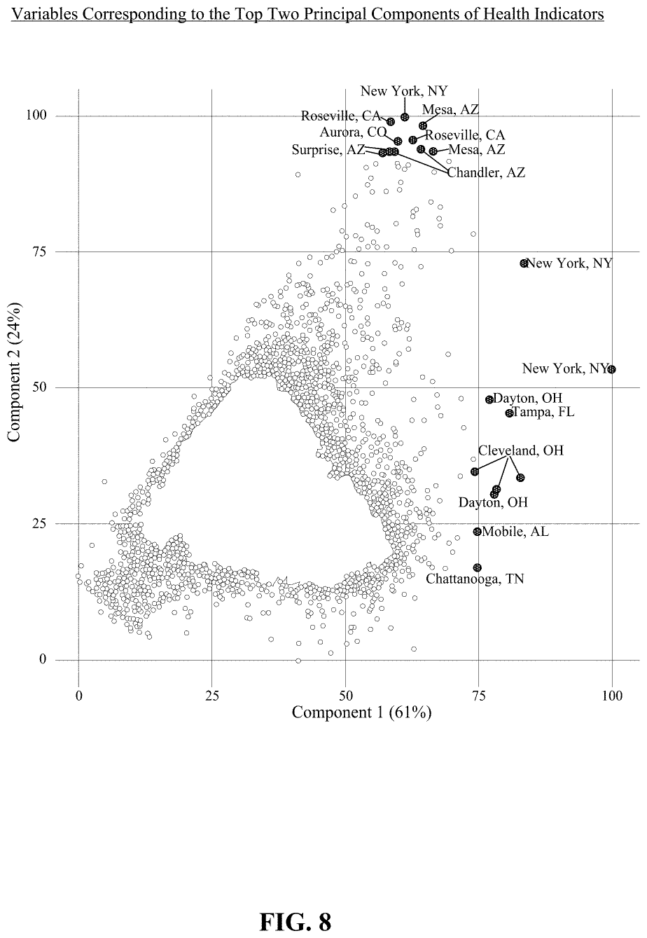

In our implementation, as presented below, two principal components of the 15 COVID-19 health indicators and risk factors described 85% of the total variation (61% and 24% for component 1 and component 2 respectively) of the variation (FIG. 8). The first 2 components expressed a high percentage of the explained variance, therefore, we selected the first 2 principal components to form basis of our new feature space. In other implementations, more than 2 principal components, up to 8 to 20 or more principal components can be selected to create a basis of Eigen images. Each production image to be analyzed by Eigen image analysis is represented as a weighted linear combination of the basis images. Each weight of the ordered set of basis components is used as a feature for training the classifier. The technology disclosed can use other image decomposition and dimensionality reduction techniques. For example, non-negative matrix factorization (NMF) which learns a parts-based representation of images as compared to PCA which learns complete representations of images. Unlike PCA, NMF learns to represent images with a set of basis images resembling parts of images.

### COVID-19 Risk Score

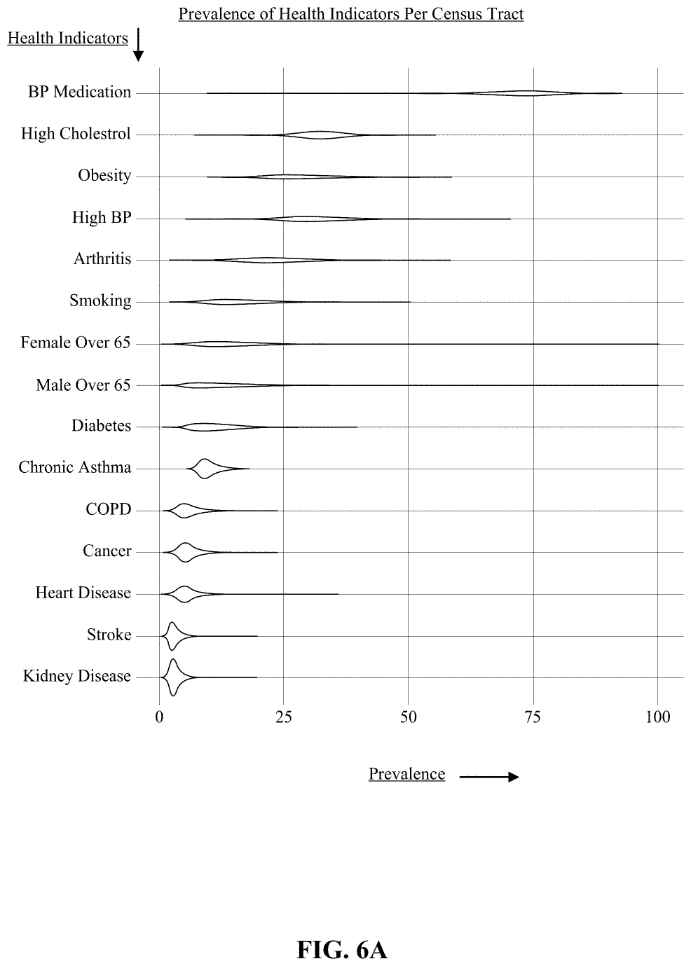

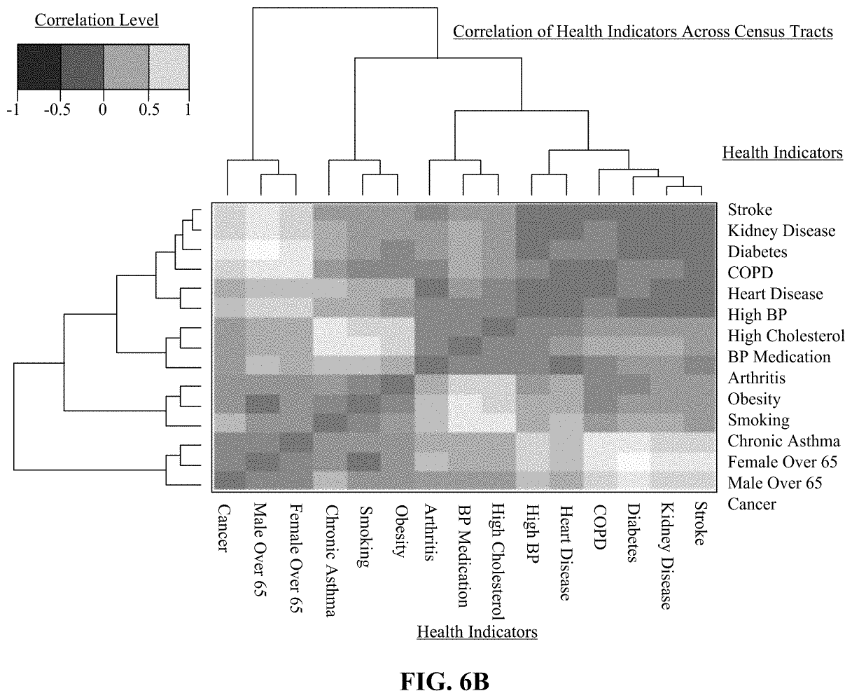

We now describe the calculation of COVID-19 risk score from selected principal components with high explained variance. We estimated the cross census-tract correlation between the 15 COVID-19 candidate health indicators and risk factors as illustrated in FIGS. 6A and 6B. We found that there is dense correlation i.e., median absolute value of correlation is 0.63 (Inter Quartile Relationship or IQR of 0.35 to 0.78) among many of the disease prevalence. Especially, higher or lower prevalence of one disease was strongly associated with higher or lower prevalence of the risk factors or complications of disease. For example, cardiometabolic diseases such as diabetes, kidney disease, stroke and heart disease displayed a strong correlation between one another, i.e., mean pairwise Pearson correlation between these 4 disease outcomes were 0.92. Risk factors for these diseases, such as obesity, high blood pressure and high cholesterol exhibited on average a correlation of 0.62 between them. Furthermore, an average correlation of 0.78 between diseases such as diabetes, kidney disease, stroke, and heart disease is observed.

Communities that had a higher prevalence of smoking also exhibited higher prevalence of chronic obstructive pulmonary disease (COPD) and chronic asthma. The correlation between asthma and COPD was 0.69; the correlation between smoking and COPD was 0.81, and between smoking and asthma was 0.78. Obesity, an established risk factor for many diseases, was correlated with all disease indicators and risk factors (mean absolute value of pairwise correlation was 0.54). Communities that had higher prevalence of males and females older than age 65 had larger prevalence of any cancer (average correlation of 0.78).

The first two principal components of the 15 COVID-19 health indicators and risk factors described 85% of the total variation (61% and 24% for component 1 and component 2 respectively) of the variation over all 29,768 census tracts. The first principal component had equal contribution from all 15 health diseases, except for cancer and males and females over the age of 65. The second principal component is dominated by cancer and age (FIG. 8). The structure across health indicators and risk factors persisted when examining on geographical units of counties and states.

We calculate the COVID-19 risk score by projecting the merged image data for 19,978 census tracts for 15 health indicators to two principal components that cumulatively represent majority of the variance (about 85%) as discussed above. Therefore, the technology disclosed reduces the merged image data from a high-dimensional merged image space to two-dimensional space based on two principal components. The COVID-19 score is weighed sum of the two variables (principal components) whereas the weight of the of respective variable is the respective variance explained by the variable (61% and 24% respectively). We scaled the score to be between 0 and 100. The average score is 33.7 with a standard deviation (SD) of 8.6. The median of the score is 33.32 and interquartile range (IQR) is 28 to 38.

Through simulations of the co-prevalence for each of the 29,978 census tracts, we found that the point estimates for the community risk scores were robust to simulated sampling error. The average error of the COVID-19 risk score across 29,978 census tracts is 1.25 (SD of 0.85).

Many cities in Southwest and Southeast United States demonstrated large disparities in the COVID-19 risk score. For example, Surprise, Ariz. had COVID-19 community scores IQR of 26 to 59. Atlanta, Ga., had an IQR of 24 to 41.

### Social Determinants of Health and COVID-19 Risk Score

“Social determinants of health” and demographic characteristics of a community explain an astounding 54% of the total additive or linear variation of the COVID-19 community risk score in the training and testing datasets, respectively. Further, it was found that an additional 11% of variation possibly attributable to non-linear relationships, or a total of 65% between social determinants and the COVID-19 community risk score in the testing data using random forest-based regression. We also found that features of the built environment captured by satellite images contributed to 27% of the variation in the COVID-10 Community Score. In total, combining both social determinants and satellite imagery explained 87% of the variation of the COVID-19 Community Score. Below, we describe the most important features of each of the models.

The 13 sociodemographic variables contribute independently to the linear association with the COVID-19 score (linear regression p-values less than 2×10⁻¹⁶ for 11 out of 13 variables). The variables that had the largest additive contribution included the proportion of the community that was non-employed. For a 1 standard deviation (SD) change in proportion of non-employed population was associated with a 5.3 unit increase in the COVID-19 score, p<2×10⁻¹⁶. Similarly, and independently, a 1 SD increase in individuals with less than a high school education was associated with a 2 unit increase in the score. However, a 1 SD change in the increase of those at or below the poverty level was associated with a 3.3 unit decrease in the COVID-19 score. There was a strong relationship between ethnic mix of a community in the COVID-19 score. For example, communities with higher prevalence of Hispanic or Asian population had an overall lower COVID-19 risk score (1 to 2 unit lower for 1 SD change) and communities with a larger African American presence had a 0.6 unit larger COVID-19 score.

We trained a random forest regressor to test for non-linear relationships (1000 trees and 4 variables at each split) in the training data. In the testing data, the social determinants of health indicators explained 65% of variation in the COVID-19 risk score in the United States. Proportion of non-employment is one of the most important variables in the training data determined through a permutation of each variable sequentially. The proportion of non-employment caused an increase of 273% in mean-squared error or MSE when permutated. Other variables such as Asian population caused an increase of 93% of MSE, percentage of population at or below poverty caused an increase of 91%, Hispanic population caused a 78% increase of MSE, and less than high school education caused 78% increase of MSE. The rank order of the importance of these variables is similar to their linear contribution.

The COVID-19 Community Risk Score can be highly correlated with indicators of the built environment as captured by satellite imagery, even after accounting for social determinants of health. We deployed existing deep learning models originally trained on images from the internet and “retrained” them to predict COVID-19 Community Risk Score. In testing data, satellite imagery information plus 13 social determinants indicators explained 87% of the COVID-19 Community Risk Score variation.

COVID-19 Risk Score Association with Death Rate in New York City

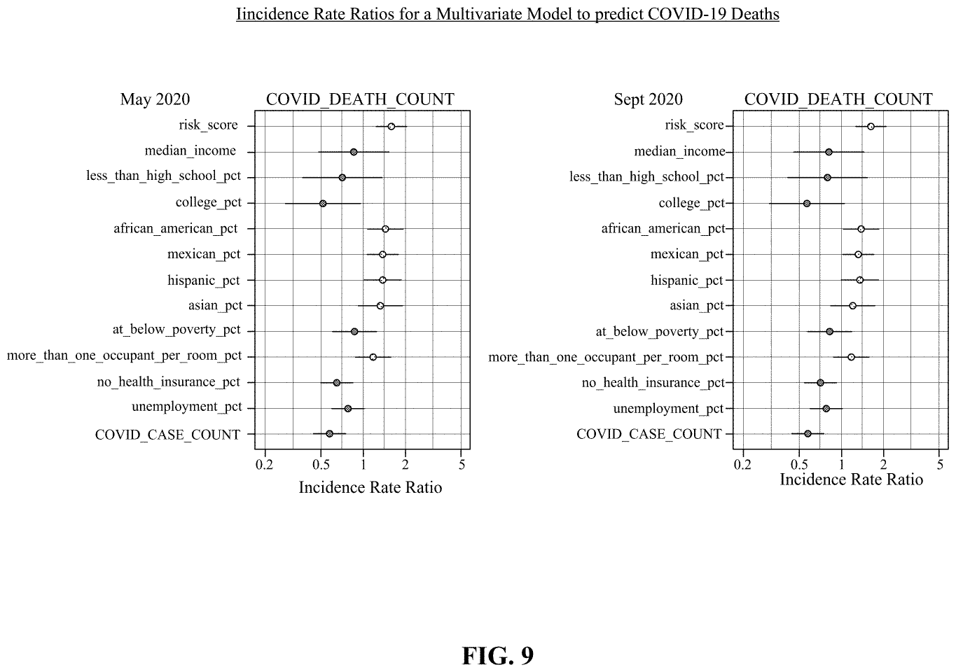

COVID-19 risk score is predictive of COVID-19 related deaths in Manhattan even after adjustment for social determinants of health as shown in FIG. 9. In brief, places with the highest COVID-19 risk score have an incidence rate ratio 2 (two folds greater than rest of the city). In FIG. 9, the graphical illustration of incidence rate ratio (IRR) for the COVID-19 Risk Score for May 2020 is presented on the left and the graphical illustration of incidence rate ratio for the COVID-19 Risk Score for September 2020 is presented on the right for comparative analysis. A rate ratio, sometimes called an incidence density ratio or incidence rate ratio, is a relative difference measure used to compare the incidence rates of events occurring at any given point in time. A common application for this measure in analytic epidemiologic studies is in the search for a causal association between a certain risk factor and an outcome.

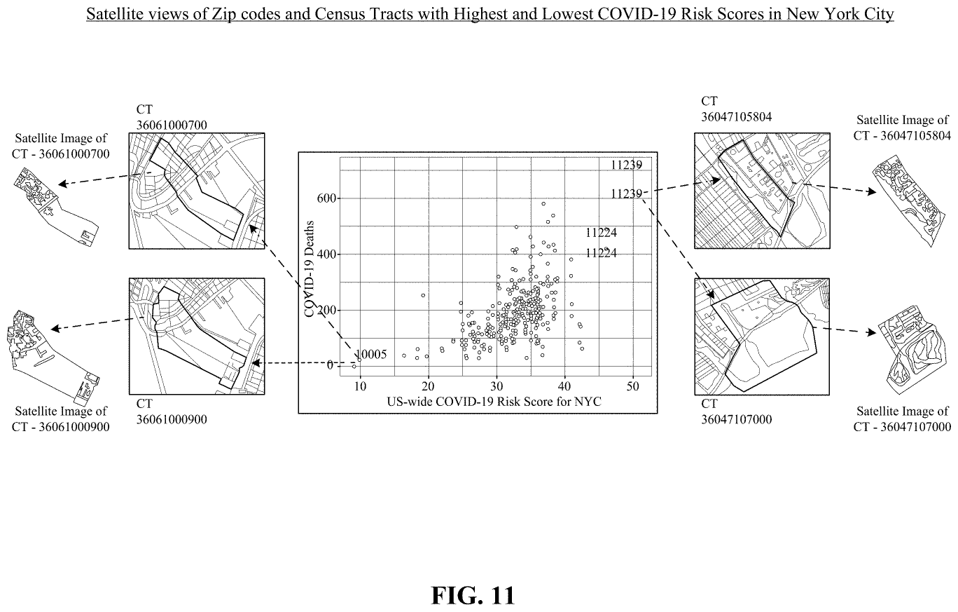

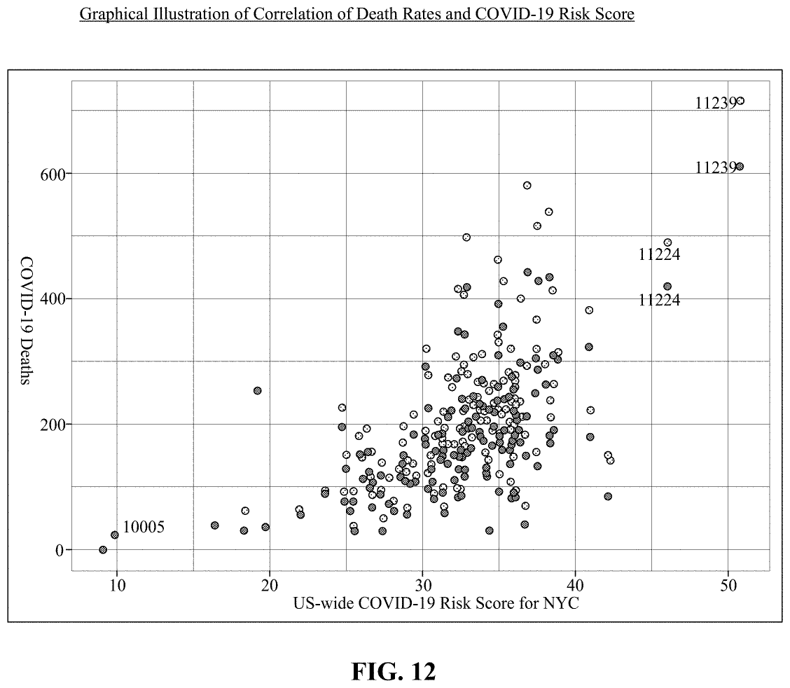

Neighborhoods with higher COVID-19 Community Risk were associated with higher rates of COVID-19 related deaths in New York City. We mapped the COVID-19 Community Risk Score to each Zip code tabulation area (ZCTA) in New York City. Each ZCTA had information on the total number of COVID-19 tests, positive cases, and COVID-19 related deaths. We associated the COVID-19 Community Risk Score with the total death count of each Zip code, adjusting for social determinants of health variables. A 1 standard deviation (SD) increase in the COVID-19 Risk Score is associated with a 39% increase in the incidence rate ratio (Incidence Rate Ratio or IRR of 1.34 per 1SD increase, pvalue<2×10⁻¹¹) as shown in FIG. 11 and FIG. 12. For Zip codes (e.g., FIG. 11 and FIG. 12 annotated Zip codes) with average COVID-19 Community Risk Scores greater than 40 had an almost 2 fold increase in death rates (IRR of 1.98, 95% CI: [1.43, 2.77], pvalue<5×10-5).

### Discussion

We present major findings before presenting details of the method.
Heterogeneity of prevalence and risk score within cities (census tract reporting where possible is greater than in counties).
Immense structure, i.e., dense correlation as first few principal components explain the COVID-19 risk.
Sociodemographic variables explain greater than 50% of variance in COVID-19 score; COVID is expected to exacerbate disparities.
The technology disclosed illustrates that it is possible to use deep learning to find signals that can predict the risk score; and can be used in future surveillance programs.
The technology disclosed includes APIs to consume publicly available health, geographic, and image databases.
The technology disclosed includes user interface elements to enable public health care decision makers to visualize and further analyze the outputs.
The technology disclosed has been applied on public health data of New York City. Similar analysis can be performed for other cities, regions, states, etc.

The technology disclosed enables this multi-scale analysis integrating information from gold standard disease prevalence sources such as the US Centers for Disease Control and Prevention, social determinants of health information from the US Census, and satellite imagery data. We demonstrate an approach to identify characteristics of communities at risk for COVID-19 complications. We use the tools of unsupervised learning to develop a COVID-19 Community Risk Score that provides a single interpretable number that summarize a communities' (census tract) aggregate risk. The constituents of the COVID-19 Risk Score are established neighborhood level risk factors for COVID-19, such as age, obesity, diabetes, and heart disease.

The COVID-19 Risk Score can be a tool in the growing armamentarium for public health and healthcare companies' toolbox to enable communities to prepare for the potential onslaught of cases, ultimately helping to “lower the curve” to achieve precision public health. We found that the Zip code-level COVID-19 Risk Score for New York City and surrounding areas predicted risk for COVID-19 complications, such as death. Zip codes with the highest COVID-19 scores (in the top 5%) had double the risk of COVID-19 death versus Zip codes with lowest scores. New York City placed a second lockdown due to a surge in COVID-19 cases in the very same Zip codes that were identified as high risk by the technology disclosed.

As an additional feature resulting from developing a risk score for communities, we observed that there is a great disparity of chronic disease prevalence within cities and across cities in the United States. With the exception of New York City and a few other places in the United States, public health agencies mostly report COVID-19 case and death records are collected at the level of the county. However, we found that that smaller populations are at risk, and counties are heterogenous.

Furthermore, it was determined that the “structure” in the disease prevalence information, or the correlation between disease prevalence was large (˜90% of the) variation of prevalence of the 15 disease and health indicators (e.g., diabetes, obesity, cardiovascular disease, populations that take blood pressure medication, and average age, among others) in the United States. It was determined that risk factors for COVID-19 can be explained by just two dimensions.

The output from technology disclosed relies on disease and health indicator prevalence from the 500 largest cities in the United States. In one implementation, the system can include satellite imaging technology for locations that cannot be covered by resource-limited public surveillance programs. Second, while the CDC 500 Cities data are reflective of the diversity of individuals who live in a Census tract, they are updated every two years and are dated to the latest collection (2019 data release reflects disease prevalence in 2017). Relatedly, individual-level disease nor COVID-19 status of individuals from these communities are measured. Third, the satellite image data are captured at a resolution of approximately 20 m per pixel. It is not clear if higher resolution images (that can theoretically capture more human-visible details of the built environment) can lead to better predictions of the COVID-19 Risk Score.

There are many social determinants of health, comprising both non-modifiable and modifiable factors, such as ethnicity, socioeconomic status, residential location, type of occupation, access to healthcare, physical exposures (e.g., air pollution and individual factors of the exposome), and built environment. They are hierarchical in structure distributed over both geographic space and time whose measurement can occur on both the individual-level (exposure of a person) or area level (exposure levels of a place). Using the technology disclosed, we performed multi-scale analysis and demonstrated how COVID-19 disease is inextricably connected to social determinants. Furthermore, we demonstrated that satellite images can provide a microscope into the area-level “built environment”, a concept that encapsulates the physical structures of how humans live, such as the city layout, resource presence, and landscape. We found that, by combining established social determinants, information measured on earth with the built environment from space can explain most of the variation in the COVID-19 Risk Score. A mere 13 sociodemographic variables can explain 50% of variation of the COVID-19 Risk Score and combined with images from space, 90% of variation in the COVID-19 Risk Score can be explained. Therefore, it is possible to survey populations where COVID-19 testing and resource allocation is overlooked through the use of our predictors.

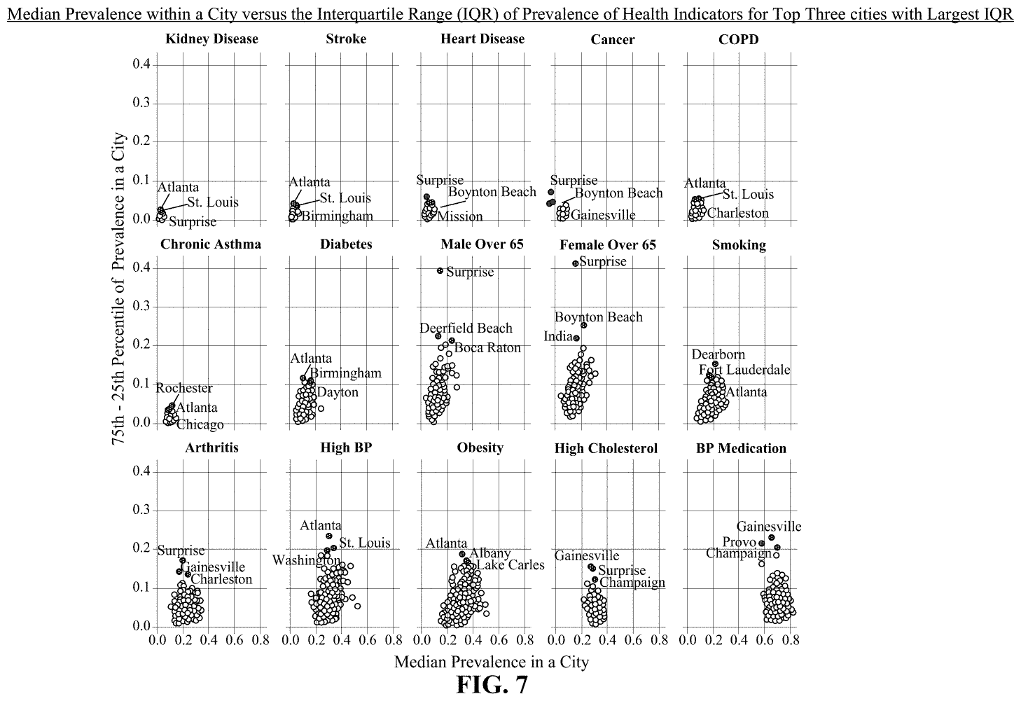

It is clear that COVID-19 is a disease of disparity; however, we cannot make a causal claim between the instruments, such as the COVID-19 Risk Score, satellite imagery, and census-tract-level sociodemographic factors and eventual individual-level COVID-19 related complications. In fact, COVID-19 may exacerbate the chronic disease disparities we observe (FIG. 6A, FIG. 6B, and FIG. 7). The technology disclosed provides tools to monitor these phenomena in real-time.

### Methods

We obtained the US Centers for Disease Control and Prevention 2017 500 Cities data (updated December 2019). The 500 Cities data contains disease and health indicator prevalence for 26,968 number individual census tracts of the 500 Cities which are estimated from the Behavioral Risk Factor Surveillance System (BRFSS).

From the 500 Cities data, we chose 13 health indicators that may put patients with COVID-19 at risk for hospitalization and death based on recent case reports emerging from Wuhan, Italy, and the United States. Disease indicators include prevalence for adults over 18 of diabetes, coronary heart disease chronic kidney disease, asthma, arthritis, any cancer, chronic obstructive pulmonary disorder. We also selected behavioral risk factors including smoking and obesity. Third, we selected variables that reflected access to care, such as prevalence of individuals on blood pressure medication and high cholesterol levels.

We obtained a 5-year 2013-2017 American Community Survey (ACS) Census data, which contains sociodemographic prevalence and median values for Census tracts. We selected demographic variables, including the total number of individuals in the tract, proportion of males and females over the age of 65, proportion of individuals by race (e.g., African American, White, Hispanic, American Indian, Pacific Islander, Asian, or Other). These data also included information on the socioeconomic indicators including median income, the proportion of individuals under poverty, unemployed, cohabitate with more than one individual per room, and have no health insurance.

### COVID-19 Community Risk Score Formulation

We merged 500 Cities' health and disease indicator prevalence for each of the 26,968 census tracts with ACS information and calculated their Pearson pairwise correlations. We considered 15 variables in total, including 13 health indicators (e.g., diseases and risk factors), and 2 demographic factors, the proportion of male and female individuals over 65 in the risk score. The disease prevalence can include diabetes, coronary heart disease, any cancer, asthma, chronic obstructive pulmonary disease, arthritis. The behavioral risk factors can include prevalence of obesity, smoking, high cholesterol and high blood pressure. The clinical risk factors included the prevalence of individuals on a blood pressure medication.

To summarize the total variation of the disease prevalence in a single score, we devised 2 similar approaches. The first approach scaled each of the 13 health indicators plus males and females over 65 (z-score transformation) prevalence by subtracting overall average and dividing by the standard deviation of the prevalence. Then, for each census tract, the z-scores were summed and rescaled to be between 0-100. Therefore, the tracts with the highest scores have the highest “additive” prevalence for all the health indicators. We call this the additive score.

The second and primary score utilized principal components analysis (PCA), a “unsupervised” machine learning approach that “reprojects” data (26,968 by 15 dimensioned in our case) into a new space where each new variable is a linear combination of variables from the original dataset. PCA attempts to maximize the variance explained over the dataset in successive new variables (call them Y1 through Y15) that are defined by the “components”, or linear combination of each of the original variables (e.g., health indicators). Therefore, the first variables (Y1, Y2, etc.) that correspond to the first principal components in the new dataset explain the maximal amount of variation in the entire dataset. After rejoining the census tracts on the first two principal components, call them Y₁ and Y₂ (fitting each census tract to the first two principal components), we ‘aligned” Y1 and Y2 such that the increasing prevalence of all the disease and health indicators were monotonically increasing with increase of the disease prevalence. Next, we estimated a single score for each census tract as a weighted average between Y1 and Y2 where the weights were proportional to the variance explained by the first and second principal components respectively. Finally, the score is rescaled to be between 0-100. The higher the value, the higher the total burden of disease and proportion of individuals over 65 in that census tract. The system can calculate risk score for different units of administrative areas, including the 500 cities, 316 counties, and 50 states. We estimated a city-wide prevalence of each of the 15 COVID-19 risk factors and diseases and then computed the additive and PCA-based scores as above. We repeated the same procedure for counties (m=316) and states (m=50).

Next, we sought to estimate how robust the PCA-based risk score is to sampling error via simulation. To do so, we estimated the standard deviation of the prevalence as a function of the size of the population of the tract. We assumed a covariance structure between the disease prevalence to be the observed census-level correlation across the US. Next, for each census tract, we simulated 100 times the prevalence of the diseases using a multivariate normal distribution, centered around the actual prevalence and with covariance equal to the COVID-19 risk factor correlation over all 26,968 tracts. Next, for each of the 100 simulated datasets we computed the principal components and obtained a simulated distribution about the principal component. Next, we estimated the predicted new projections for plus or minus 5 standard deviations (SD) of the principal components. The “robustness” score is the range of the score across the +/−5 SD of the principal components.

Reconstructing the COVID-19 Community Risk Score from Satellite Imagery

To reconstruct the COVID-19 risk score from satellite imagery, millions of satellite images (n=4,742,919) are analyzed in an ensemble of an unsupervised deep learning algorithm and a supervised machine learning algorithm. The images are satellite raster tiles that can be downloaded from the OpenMapTiles database or other satellite image databases. The images have a spatial resolution close to 20 meters per pixel allowing a maximum zoom level of 13. Images are extracted in tiles from the OpenMapTiles database using the coordinate geometries of the census tracts. After extraction, images are digitally enlarged to achieve a zoom level of 18. It is understood that the technology disclosed can be applied to other zoom levels greater than or less than zoom level of 18.

Many census tracts are large enough to contain multiple satellite images. The median number of images per tract is n=94, and the number of images per census tract ranges from n=1 image in the census tract to the largest geographical tract with n=162,811 images (in Anchorage, Ak.) with an interquartile range from 43 to 182 images. The geographical coverage of the images per census tract ranges from the smallest census tract covering 0.022 km² and the largest census tract covering 5,679.52 km², with an interquartile range from 0.93 km² to 3.89 km² and a median of 1.92 km² per census tract.

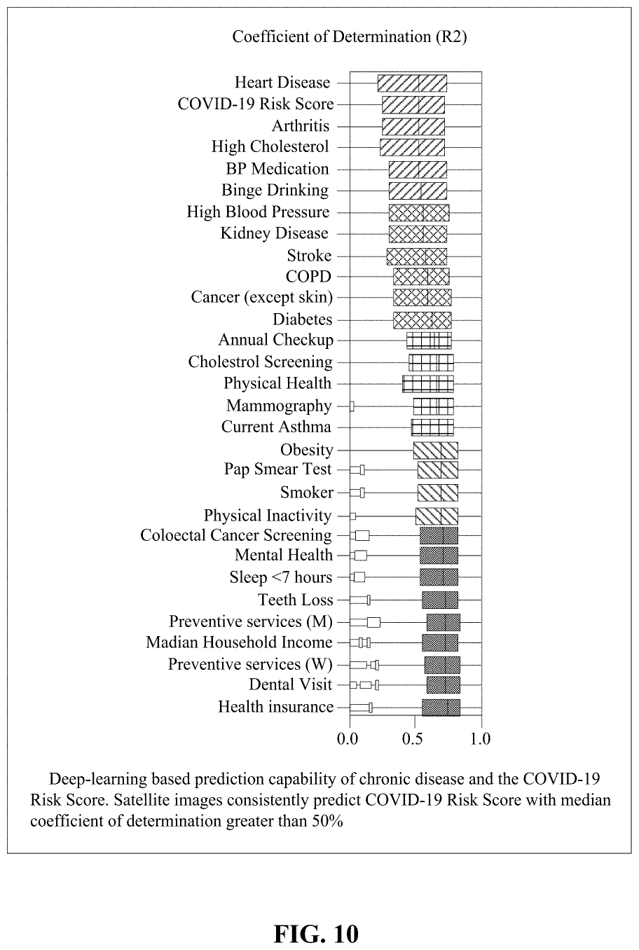

First, we passed images through AlexNet, a pretrained convolutional neural network, in an unsupervised deep learning approach called feature extraction. The resulting vector from this process is a “latent space feature” representation of the image comprising 4,096 features. This latent space representation is an encoded (non-human readable) version of the visual patterns found in the satellite images, which, when coupled with machine learning approaches, is used to model the built environment of a given census tract. For each census tract, the system calculates the mean of the latent space feature representation. The system then performs feature extraction. In one implementation, feature extraction can be performed using an NVIDIA Tesla T4 GPU using Python 3.7.7 and the PyTorch package. Finally, the latent space feature representation is regressed against the COVID-19 Risk Score using gradient boosted decision trees. We report coefficient of determination (R2) for the predictions in the test data in FIG. 10 which illustrates deep learning-based prediction capability of chronic disease and the COVID-19 Risk Score. Satellite images consistently predict COVID-19 Risk Score with median coefficient of determination (R2) greater than 50%.

In one implementation, XGBoost machine learning model is trained and applied during inference. We split the 80% of the data into training and the remaining 20% to testing. To train the model, we used a maximum tree depth of 5, a subsample of 80% of the features per tree, a learning rate (i.e., feature weight shrinkage for each boosting step) of 0.1, and used 3-fold cross-validation to determine the optimal number of boosted trees. In one implementation, training is completed on a NVIDIA Tesla T4 GPU using Python 3.7.7 and the XGBoost package. In a separate analysis, both satellite image features and the same social determinants of health features (above) were regressed against the COVID-19 Risk Score.

### Socioeconomic Correlates of the COVID-19 Community Risk Score

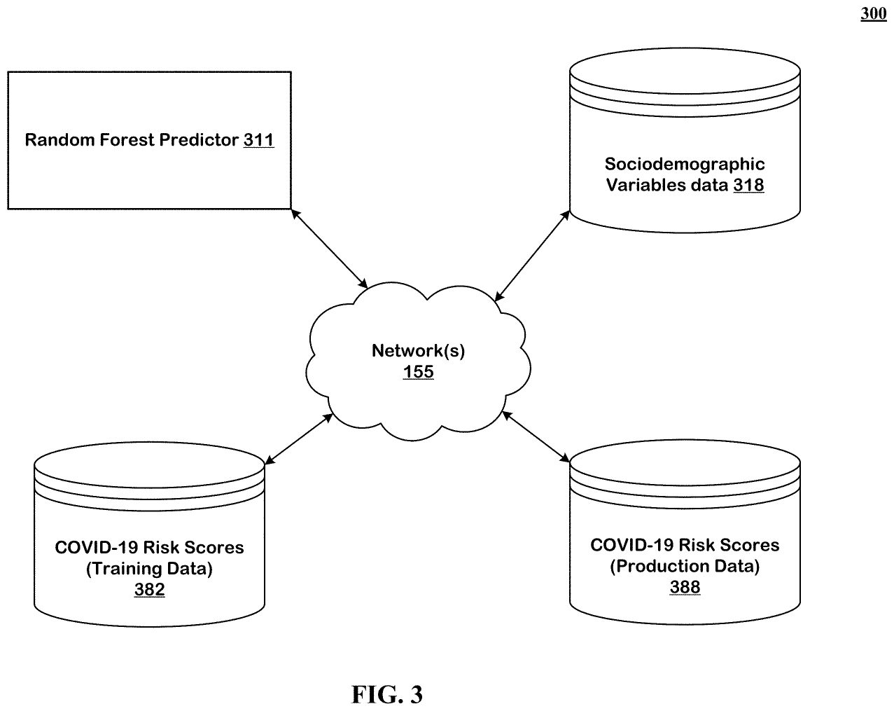

FIG. 3 is an architectural level schematic of a system 300 for predicting impact of sociodemographic variables on COVID-19 risk score. The system includes a random forest predictor 311, a sociodemographic variables database 318, COVID-19 risk score training data 382 and COVID-19 risk score production data 388. The above listed system components are in communication with each other via a network(s) 155. The technology disclosed can predict a level of impact of a plurality of sociodemographic variable on COVID-19 risk score for census tract-level communities using a trained random forest regressor 311.

Random forest classifier (also referred to as random decision forest) is an ensemble machine learning technique. Ensembled techniques or algorithms combine more than one technique of the same or different kind for classifying objects. The random forest classifier consists of multiple decision trees that operate as an ensemble. Each individual decision tree in random forest acts as base classifier and outputs a class prediction. The class with the most votes becomes the random forest model's prediction. The fundamental concept behind random forests is that a large number of relatively uncorrelated models (decision trees) operating as a committee will outperform any of the individual constituent models. In the following sections, we present further details of training and production modes of the random forest regressor.

### Training of Random Forest Classifiers

The system can train a random forest regressor for predicting a level of impact of a plurality of sociodemographic variables on COVID-19 risk score for census tract-level communities. The system can use labeled training examples of COVID-19 risk score per census tract-level community as input to the random forest regressor. The system can train the random forest regressor using features of the COVID-19 risk score for one-vs-the-rest determination of the plurality of sociodemographic variables of the labeled training examples. The system can store parameters of the trained random forest regressor for use in production mode to determine the level of impact of the plurality of sociodemographic variables on COVID-19 risk score for census tract-level communities.

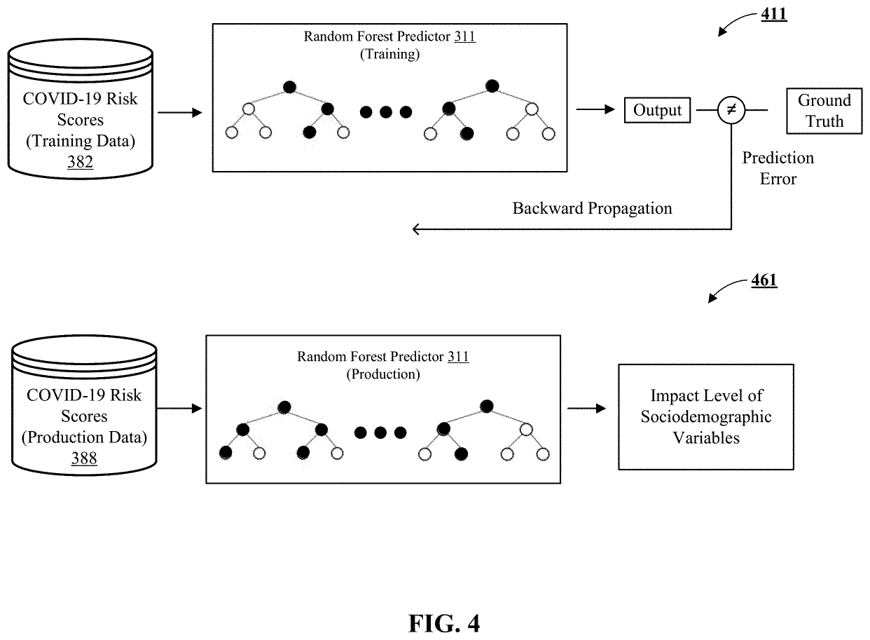

FIG. 4 present an illustration 411 showing training of a random forest regressor. The use of random forest regressor in production is shown as illustration 461 in FIG. 4.

The training data comprises of input features for the labeled satellite image data merged with chronic disease prevalence data in census tract-level communities. The size of the training database 382 is expected to grow as more labeled satellite images merged with chronic disease prevalence are added to the training data set.

A random forest classifier with 1000 decision trees and a depth of 4 worked well. It is understood that random forest classifiers with a range of 500 to 1500 decision trees and a range of depth from 4 to 10 is expected to provide good results for this implementation. We tuned the hyperparameters using randomized search cross-validation. Increasing the number of trees can increase the performance of the model however, it can also increase the time required for training.

Decision trees are prone to overfitting. To overcome this issue, bagging technique is used to train the decision trees in random forest. Bagging is a combination of bootstrap and aggregation techniques. In bootstrap, during training, we take a sample of rows from our training database and use it to train each decision tree in the random forest. For example, a subset of features for the selected rows can be used in training of decision tree 1. Therefore, the training data for decision tree 1 can be referred to as row sample 1 with column sample 1 or RS1+CS1. The columns or features can be selected randomly. The decision tree 2 and subsequent decision trees in the random forest are trained in a similar manner by using a subset of the training data. Note that the training data for decision trees is generated with replacement i.e., same row data can be used in training of multiple decision trees.

The second part of bagging technique is the aggregation part which is applied during production. Each decision tree outputs a classification for each class. In case of binary classification, it can be 1 or 0. The output of the random forest is the aggregation of outputs of decision trees in the random forest with a majority vote selected as the output of the random forest. By using votes from multiple decision trees, a random forest reduces high variance in results of decision trees, thus resulting in good prediction results. By using row and column sampling to train individual decision trees, each decision tree becomes an expert with respect to training records with selected features.

During training, the output of the random forest is compared with ground truth labels and a prediction error is calculated. During backward propagation, the weights of the components are adjusted so that the prediction error is reduced. The number of components depends on the number of components selected from output of principal component analysis (PCA) using the explained variance measure. During regressor, the system can use features of the COVID-19 risk score and apply one-vs-the-rest (OvR) determination of the plurality of sociodemographic variables of the labeled training examples. The parameters (such as weights of components) of the trained random forest classifier are stored for use in production classification of sociodemographic variables.

### Prediction Using Random Forest Classifiers

In a production or inference mode, the system can predict a level of impact of a plurality of sociodemographic variables on COVID-19 risk score for census tract-level communities using a trained random forest regressor. The system can use production examples of COVID-19 risk score per census tract-level community as input to the trained random forest regressor. The system can produce an additive contribution of each of the plurality of sociodemographic variables on COVID-19 risk score for census tract-level communities. The system can provide the additive contributions of the plurality of sociodemographic variable for use in detection of sociodemographic variables with high additive contributions to the COVID-19 risk score.

We now describe the prediction of the level of impact of sociodemographic variables using the trained classifier 311 shown in the illustration 461 in FIG. 4. The process starts when production COVID-19 risk scores are provided as input to the trained random forest regressor or predictor 311. The regressor can predict impact level for each of the sociodemographic variables on the COVID-19 risk score. Therefore, each decision tree in the random forest will output thirteen probability values, i.e., one value per sociodemographic variable. The results from the decision trees are aggregated and majority vote is used to predict impact level of sociodemographic variable.

We associated each of the ACS-established sociodemographic indicators with the COVID-19 community risk score multivariate linear and random forests regression to test the linear and non-linear contribution of the sociodemographic indicators in the COVID-19 Score. We split the dataset into half “training” and “testing” to get a conservative estimate of variance explained and predictive capability of the sociodemographic variables in the COVID-19 risk score while not overfitting the data. Specifically, we tested the linear and non-linear association or prediction between American Community Survey 5-year proportions of individuals in each census tract who were (a) below poverty, (b) unemployed, (c) non-employed, (d) have less than high school education, (e) lack health insurance, (f) have more than one person occupied per room, and (g) Hispanic, Asian, African American or Other Ethnic group. Furthermore, we also included the median income and median home value of a census tract. Coefficients in the linear regression denoted a 1 unit change in a 1-unit change in the socioeconomic variable (e.g., a 1 SD unit change in the prevalence or a 1 SD change in the median income for a census tract). Random forests were fit using 100 trees and a tree size of 5.

Association of COVID-19 Community Risk Score with Zip code-level COVID-19 Attributed Mortality

The system can download case and death count data on a Zip code tabulation area (ZCTA) of New York City, a hotspot of the US COVID-19 epidemic as of May 20, 2020. We used 2010 Census cross-over files to map Census tracts to ZCTAs. The system can compute the average COVID-19 Community Risk Score of the ZCTA, weighting the average by population size of the Census tract. Like the above, we estimated the ZCTA-level socioeconomic values and proportions. The system includes logic to associate the COVID-19 with the death rate using a negative binomial model. In one implementation the offset term is set as the logarithm of the total population size of a Zip code. The exponentiated coefficients are interpreted as the incidence rate ratio for a unit change in the variable (versus no change).

### Visualizing the COVID-19 Community Risk Score

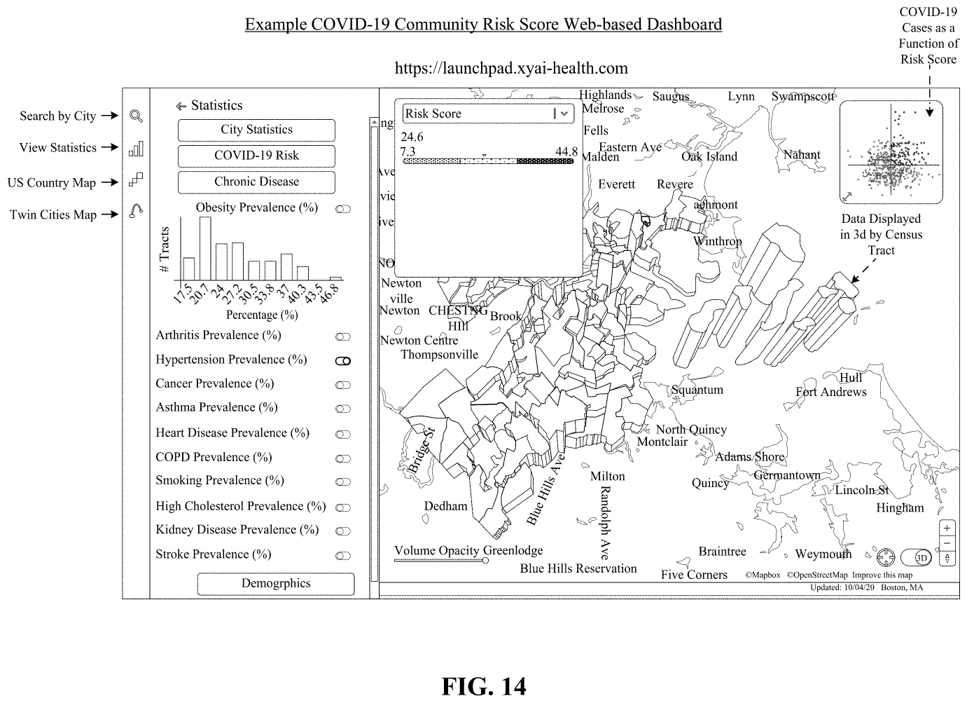

The system can merge each of the 26,968 with geometry shape files from the Census. These shape files can be plotted as a choropleth visualization (FIG. 14), where the color of each census tract depicts the value of the nation-wide COVID-19 Risk Score. Using the system, we can visualize dynamic SARS-CoV-19 testing rates, positive tests, and death rates with information sourced from Johns Hopkins University. Other sources of data can be used for the above visualization. The system can also acquire information on the number of hospital beds across the United States, as well as intensive care unit beds from Kaiser News. Finally, using the system we can visualize the variables across all of the 26,968 tracts with Uniform Manifold Approximation and Projection.

### The Community COVID-19 Risk Score Application Programming Interface

To distribute the data to other software applications, users, and provide interactive graphing capabilities, an Application Programming Interface (API) and dashboard for the COVID-19 Community Risk Score is developed (FIG. 14). The system presents information about hospital beds from the Homeland Infrastructure Foundation-Level Data (available at <<hifld-geoplatform.opendata.arcgis.com/datasets/hospitals>>) and ICU beds from the Kaiser Family Foundation (available at <<kfforg/global-health-policy/press-release/new-khn-reporting-reveals-half-of-nations-counties-lack-intensive-care-beds-as-covid-19-cases-rapidly-increase/>>). The system can obtain COVID-19 case data from Johns Hopkins University or other data sources or databases. The system indexes the COVID-19 Risk Score by Census tract FIPS code (or the county code when appropriate) and stores in a PostGIS-enabled Postgres database v3.0.1 (available at <<postgis.net/>>). We also developed a dashboard through the API in the form of geographical coordinate information for Census tracts, and data values (in GeoJSON format) to construct a choropleth in the web browser (FIG. 14) using the Mapbox mapping library v1.11.0 (available at <<mapbox.com/>>) and Chart.js v2 (available at <<chartjs.org>>). The system obtains geographical coordinates and shapefiles from the US Census 2018, (available at <<www.census.gov/geographies/mapping-files/time-series/geo/carto-boundary-file.html>>). Distributions of values are displayed for Census tracts being viewed at a particular location. The interactive dashboard provided by the technology disclosed is illustrated in FIG. 14 (available at <<https://dashboard.xyai-health.com/anthem/11>>).

### Particular Implementations

We describe implementations of a system for chronic disease outcome for communities at risk for COVID-19 related complications e.g., death, ventilator use, hospitalization, etc.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A system implementation of the technology disclosed includes one or more processors coupled to memory. The memory can be loaded with instructions to predict chronic disease outcome for census tract-level communities at risk for COVID-19 related complications. The system includes logic to access satellite image data of built environment for census tract-level communities. The system can merge image data for census tract-level communities with respective chronic disease prevalence data per census tract-level community for a plurality of chronic diseases. The merging of data results in merged image data in high-dimensional image space. The system can identify principal components forming a basis of the high-dimensional image space. An image in the high-dimensional image space can be represented as a linear combination of the principal components.

The system includes logic to select a subset of the principal components to project high-dimensional merged image data to a low-dimensional feature subspace by selecting a subset of the principal components. The system selects principal components that cumulatively describe at least fifty percent of explained variance. Explained variance indicates how much information (or variance) can be attributed to a principal component. The system can calculate a COVID-19 risk score for census tract-level communities as a weighted combination of the selected principal components. The COVID-19 risk score predicts risk for COVID-19 related complications as a function of chronic disease outcome. The system includes logic to provide COVID-19 risk score to a public health policy decision maker for use in public health policy decisions for census tract-level communities.

This system implementation and other systems disclosed optionally include one or more of the following features. System can also include features described in connection with methods disclosed. In the interest of conciseness, alternative combinations of system features are not individually enumerated. Features applicable to systems, methods, and articles of manufacture are not repeated for each statutory class set of base features. The reader will understand how features identified in this section can readily be combined with base features in other statutory classes.

In one implementation, the plurality of chronic diseases can be grouped in at least three categories including cardiometabolic diseases, cancerous diseases and joint inflammation diseases (arthritis). The cardiometabolic diseases can include at least one of diabetes, kidney disease, stroke, and heart disease. In other implementation, the system can include other grouping of diseases in which chronic diseases can be grouped in more than three groups. In one implementation, the system can include logic to merge the image data for census tract-level communities with respective behavioral risk factor data per census tract-level community for a plurality of risk factors. The merging can produce merged image data in high-dimensional image space. The risk factors can include at least one of smoking and obesity.

In one implementation, at least one principal component representing contribution from cardiometabolic diseases describes at least fifty percent of the explained variance.

In one implementation, at least one principal component representing contribution from cancerous diseases for persons above age 65 describes at least twenty percent of the explained variance.

In one implementation of the technology disclosed, the system can include logic to train a random forest regressor for predicting a level of impact of a plurality of sociodemographic variables on COVID-19 risk score for census tract-level communities. In the training implements, the system can include using labelled training examples of COVID-19 risk score per census tract-level community as input to the random forest regressor. The system includes logic to train the random forest regressor using features of the COVID-19 risk score for one-vs-the-rest determination of the plurality of sociodemographic variables of the labelled training examples. The system can store parameters of the trained random forest regressor for use in production of the level of impact of the plurality of sociodemographic variables on COVID-19 risk score for census tract-level communities. In such implementation, the sociodemographic variables can include at least one of non-employed persons, persons with less than high school education, persons at or below poverty level, persons with lack of health insurance, rooms occupied with more than one person and ethnic mix of a community.

In one implementation, the system can predict a level of impact of a plurality of sociodemographic variables on COVID-19 risk score for census tract-level communities using a trained random forest regressor. The system can use production examples of COVID-19 risk score per census tract-level community as input to the trained random forest regressor. The system can produce an additive contribution of each of the plurality of sociodemographic variables on COVID-19 risk score for census tract-level communities. The system can provide the additive contributions of the plurality of sociodemographic variable for use in detection of sociodemographic variables with high additive contributions to the COVID-19 risk score.

In such implementation, the random forest regressor includes decision trees in a range of 500 to 1500 decision trees. The decision trees in the random forest regressor have a depth in a range of 4 to 10.

Other implementations consistent with this system may include a non-transitory computer readable storage medium storing instructions executable by a processor to perform functions of the system described above. Yet another implementation may include a method performing the functions of the system described above.

Aspects of the technology disclosed can be practiced as a method of predicting chronic disease outcome for census tract-level communities at risk for COVID-19 related complications (e.g., death, ventilator use, hospitalization, etc.). The method can include accessing satellite image data of built environment for census tract-level communities. The method can include merging the image data for census tract-level communities with respective chronic disease prevalence data per census tract-level community for a plurality of chronic diseases resulting in merged image data in high-dimensional image space. The method can include identifying principal components forming a basis of the high-dimensional image space. An image in the high-dimensional image space can be represented as a linear combination of the principal components. The method can include selecting a subset of the principal components to project high-dimensional merged image data to a low-dimensional feature subspace by selecting a subset of the principal components that cumulatively describe at least fifty percent of explained variance. The explained variance can indicate how much information (or variance) can be attributed to a principal component. The method can include calculating a COVID-19 risk score for census tract-level communities as a weighted combination of the selected principal components. The COVID-19 risk score can predict risk for COVID-19 related complications as a function of chronic disease outcome. The method can provide COVID-19 risk score to a public health policy decision maker for use in public health policy decisions for census tract-level communities.

This method implementation can incorporate any of the features of the system described immediately above or throughout this application that apply to the method implemented by the system. In the interest of conciseness, alternative combinations of method features are not individually enumerated. Features applicable to systems, methods, and articles of manufacture are not repeated for each statutory class set of base features. The reader will understand how features identified in this section for one statutory class can readily be combined with base features in other statutory classes.

Other implementations consistent with this method may include a non-transitory computer readable storage medium storing instructions executable by a processor to perform any of the methods described above. Yet another implementation may include a system with memory loaded from a computer readable storage medium with program instructions to perform the method described above. The system can be loaded from either a transitory or a non-transitory computer readable storage medium.

As an article of manufacture, rather than a method, a non-transitory computer readable medium (CRM) can be loaded with program instructions executable by a processor. The program instructions when executed, implement the computer-implemented method described above. Alternatively, the program instructions can be loaded on a non-transitory CRM and, when combined with appropriate hardware, become a component of one or more of the computer-implemented systems that practice the method disclosed.

Each of the features discussed in this particular implementation section for the method implementation apply equally to CRM implementation. As indicated above, all the method features are not repeated here, in the interest of conciseness, and should be considered repeated by reference.

### Computer System

FIG. 5 is a simplified block diagram of a computer system 500 that can be used to implement the technology disclosed. Computer system typically includes at least one processor 572 that communicates with a number of peripheral devices via bus subsystem 555. These peripheral devices can include a storage subsystem 510 including, for example, memory subsystem 522 and a file storage subsystem 536, user interface input devices 538, user interface output devices 576, and a network interface subsystem 574. The input and output devices allow user interaction with computer system. Network interface subsystem provides an interface to outside networks, including an interface to corresponding interface devices in other computer systems.

User interface input devices 538 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

User interface output devices 576 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system to the user or to another machine or computer system.

Storage subsystem 510 stores programming and data constructs that provide the functionality of some or all of the modules and methods described herein. These software modules are generally executed by processor alone or in combination with other processors.

Memory used in the storage subsystem can include a number of memories including a main random access memory (RAM) 532 for storage of instructions and data during program execution and a read only memory (ROM) 534 in which fixed instructions are stored. The file storage subsystem 536 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem in the storage subsystem, or in other machines accessible by the processor.

Bus subsystem 555 provides a mechanism for letting the various components and subsystems of computer system communicate with each other as intended. Although bus subsystem is shown schematically as a single bus, alternative implementations of the bus subsystem can use multiple busses.

Computer system itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system depicted in FIG. 5 is intended only as a specific example for purposes of illustrating the technology disclosed. Many other configurations of computer system are possible having more or less components than the computer system depicted in FIG. 5.

The computer system 500 includes GPUs or FPGAs 578. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft’ Stratix V FPGA, Graphcore's Intelligent Processor Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

We claim as follows:

1. A method of predicting chronic disease outcome for census tract-level communities at risk for COVID-19 related complications, the method including:
accessing satellite image data of built environment for census tract-level communities;
merging the image data for census tract-level communities with respective chronic disease prevalence data per census tract-level community for a plurality of chronic diseases resulting in merged image data in high-dimensional image space;
identifying principal components forming a basis of the high-dimensional image space wherein an image in the high-dimensional image space is represented as a linear combination of the principal components;
selecting a subset of the principal components to project the high-dimensional merged image data to a low-dimensional feature subspace by selecting a subset of the principal components that cumulatively describe at least fifty percent of explained variance wherein the explained variance indicates how much information can be attributed to a principal component;
calculating a COVID-19 risk score for census tract-level communities as a weighted combination of the selected subset of principal components, wherein the COVID-19 risk score predicts risk for COVID-19 related complications as a function of chronic disease outcome; and
providing the COVID-19 risk score to a public health policy decision maker for use in public health policy decisions for census tract-level communities.

2. The method of claim 1, wherein the plurality of chronic diseases are grouped in at least three categories including cardiometabolic diseases, cancerous diseases and joint inflammation diseases.

3. The method of claim 2, wherein the cardiometabolic diseases include at least one of diabetes, kidney disease, stroke, and heart disease.

4. The method of claim 1, further including:
merging the image data for census tract-level communities with respective behavioral risk factor data per census tract-level community for a plurality of risk factors resulting in merged image data in high-dimensional image space.

5. The method of claim 4, wherein the risk factors include at least one of smoking and obesity.

6. The method of claim 1, wherein at least one principal component representing contribution from cardiometabolic diseases describes at least fifty percent of the explained variance.

7. The method of claim 1, wherein at least one principal component representing contribution from cancerous diseases for persons above age 65 describes at least twenty percent of the explained variance.

8. The method of claim 1, further including:
training a random forest regressor for predicting a level of impact of a plurality of sociodemographic variables on COVID-19 risk score for census tract-level communities, the method including:
using labelled training examples of COVID-19 risk score per census tract-level community as input to the random forest regressor;
training the random forest regressor using features of the COVID-19 risk score for one-vs-the-rest determination of the plurality of sociodemographic variables of the labelled training examples; and
storing parameters of the trained random forest regressor for use in production of the level of impact of the plurality of sociodemographic variables on COVID-19 risk score for census tract-level communities.

9. The method of claim 8, wherein the sociodemographic variables include at least one of non-employed persons, persons with less than high school education, persons at or below poverty level, persons with lack of health insurance, rooms occupied with more than one person and ethnic mix of a community.

10. The method of claim 1, further including:
predicting a level of impact of a plurality of sociodemographic variables on COVID-19 risk score for census tract-level communities using a trained random forest regressor, the method including:
using production examples of COVID-19 risk score per census tract-level community as input to the trained random forest regressor;
producing an additive contribution of each of the plurality of sociodemographic variables on COVID-19 risk score for census tract-level communities; and
providing the additive contributions of the plurality of sociodemographic variable for use in detection of sociodemographic variables with high additive contributions to the COVID-19 risk score.

11. The method of claim 10, wherein the random forest regressor includes decision trees in a range of 500 to 1500 decision trees.

12. The method of claim 10, wherein decision trees in the random forest regressor have a depth in a range of 4 to 10.

13. A system including one or more processors coupled to memory, the memory loaded with computer instructions to predict chronic disease outcome for census tract-level communities at risk for COVID-19 related complications, the instructions, when executed on the processors, implement actions comprising:
accessing satellite image data of built environment for census tract-level communities;
merging the image data for census tract-level communities with respective chronic disease prevalence data per census tract-level community for a plurality of chronic diseases resulting in merged image data in high-dimensional image space;
identifying principal components forming a basis of the high-dimensional image space wherein an image in the high-dimensional image space is represented as a linear combination of the principal components;
selecting a subset of the principal components to project the high-dimensional merged image data to a low-dimensional feature subspace by selecting a subset of the principal components that cumulatively describe at least fifty percent of explained variance wherein the explained variance indicates how much information can be attributed to a principal component;
calculating a COVID-19 risk score for census tract-level communities as a weighted combination of the selected subset of principal components, wherein the COVID-19 risk score predicts risk for COVID-19 related complications as a function of chronic disease outcome; and
providing the COVID-19 risk score to a public health policy decision maker for use in public health policy decisions for census tract-level communities.

14. The system of claim 13, wherein the plurality of chronic diseases are grouped in at least three categories including cardiometabolic diseases, cancerous diseases and joint inflammation diseases.

15. The system of claim 14, wherein the cardiometabolic diseases include at least one of diabetes, kidney disease, stroke, and heart disease.

16. The system of claim 13, further implementing actions comprising:
merging the image data for census tract-level communities with respective behavioral risk factor data per census tract-level community for a plurality of risk factors resulting in merged image data in high-dimensional image space.

17. The system of claim 16, wherein the risk factors include at least one of smoking and obesity.

18. The system of claim 13, wherein at least one principal component representing contribution from cardiometabolic diseases describes at least fifty percent of the explained variance.

19. The system of claim 13, wherein at least one principal component representing contribution from cancerous diseases for persons above age 65 describes at least twenty percent of the explained variance.

20. The system of claim 13, further implementing actions comprising:
training a random forest regressor for predicting a level of impact of a plurality of sociodemographic variables on COVID-19 risk score for census tract-level communities, including:
using labelled training examples of COVID-19 risk score per census tract-level community as input to the random forest regressor;
training the random forest regressor using features of the COVID-19 risk score for one-vs-the-rest determination of the plurality of sociodemographic variables of the labelled training examples; and
storing parameters of the trained random forest regressor for use in production of the level of impact of the plurality of sociodemographic variables on COVID-19 risk score for census tract-level communities.
